(19) United States
(12) Patent Application Publication
(10) Pub. No.: US 2004/0037837 A1
(43) Pub. Date: Feb. 26, 2004

(54) Mycobacterium ag85 complex-specific t cell peptides and use in diagnostic and therapeutic applications thereof

(76) Inventors: Hazel Marguerite Dockrell, London (GB); Steven Mark Smith, Cornwall (GB); Roger Brookes, Gambia (ZA)

Correspondence Address: NIXON & VANDERHYE, PC, 1100 N GLEBE ROAD, 8TH FLOOR, ARLINGTON, VA, 22201-4714, US

(21) Appl. No.: 10/203,562

(22) PCT Filed: Feb. 12, 2001
(86) PCT No.: PCT/GB01/00561

(30) Foreign Application Priority Data
Feb. 10, 2000 (GB) 00030825

(51) Int. Cl. A61K 39/00; A61K 39/38

(52) U.S. Cl. 424/184.1

(57) ABSTRACT

Use of (a) a polypeptide which comprises an epitope sequence of the formula (I): X₁ L/I/M X₂ X₃ X₄ X₅ X₆ X₇ V/L/I wherein X₁ is G, X₂ is P, X₃ is V, X₄ is E, X₅ is Y, X₆ is L and X₇ is Q, or X₁ is K or R, X₂ is I or V, X₃ is A, X₄ is N, X₅ is N, X₆ is T and X₇ is R, or an epitope sequence which is an analogue of (I) and which can be recognised by a CD8 T cell that recognises (I); or (b) an expression vector comprising a polynucleotide encoding a said polypeptide (a) operably linked to a regulatory sequence capable of providing for expression of the said polypeptide (a); for use in the manufacture of a medicament for vaccinating prophylactically or therapeutically against infection by a mycobacterium by stimulating a CD8 T cell response. Also provided are polypeptides, expression vectors and cells which can be used to treat mycobacterial infections; and a method of detecting T cells that can recognise the epitope defined above.

### FIELD OF THE INVENTION

The invention relates to prophylactic or therapeutic vaccination against mycobacterial infection and to polynucleotides and polypeptides which can be used for such vaccination.

### BACKGROUND OF THE INVENTION

Mycobacterium tuberculosis, the causative agent of human tuberculosis, remains a major public health risk world-wide. The only vaccine currently available for human use against the disease is M. bovis bacillus Calmette-Guerin (BCG). However, its efficacy is variable and limited, especially against pulmonary tuberculosis. For a vaccine to be effective it must cause the generation of a strong immune response against M. tuberculosis.

The cellular arm of the immune response mediated by CD4 T cells has been established as an essential component of the protective immune response against M. tuberculosis. However, few mycobacterial antigens have been shown to posses CD8 T cell epitopes which are recognised by humans.

### SUMMARY OF THE INVENTION

The inventors have found epitopes in the mycobacterial antigen 85A (Ag85A) protein which cause the generation of strong CD8 T cell response in humans. The CD8 T cells of the response are present in frequencies which are comparable to that obtained with a flu matrix protein epitope known to generate an unusually strong response. Before CD8 T cell responses can be characterised using in vitro assays it is normally necessary to culture them in the presence of antigen (in vitro restimulation for 5 to 7 days). However the CD8 T cell response to the epitopes found by the inventors do not require such restimulation before being characterised in ELISPOT assays. The inventors have also shown that the CD8 T cells which recognise the epitopes are able to lyse macrophages infected with live M. tuberculosis.

Accordingly the invention provides use of (a) a polypeptide which comprises an epitope sequence of formula (I):

X₁ L/I/M X₂ X₃ X₄ X₅ X₆ X₇ V/L/I (I)

wherein

X₁ is G, X₂ is P, X₃ is V, X₄ is E, X₅ is Y, X₆ is L and X₇ is Q, or

X₁ is K or R, X₂is I or V, X₃is A, X₄ is N, X₅ is N, X₆ is T and X₇ is R, or an epitope sequence which is an analogue of (I) and which can be recognised by a CD8 T cell that recognises (I); or

(b) an expression vector comprising a polynucleotide encoding a said polypeptide (a) operably linked to a regulatory sequence capable of providing for expression of the said polypeptide (a);

for use in the manufacture of a medicament for vaccinating prophylactically or therapeutically against infection by a mycobacterium by stimulating a CD8 T cell response.

The invention further provides a vaccine composition which comprises a polypeptide or expression vector as defined above and an adjuvant or delivery system capable of stimulating a CD8 T cell response. Also provided are:

a method of vaccinating a pre-selected host to stimulate a CD8 T cell response against a mycobacterial infection, comprising administering to the host an effective amount of a polypeptide, expression vector or vaccine composition as defined above;

specific novel polypeptides, expression vectors and cells, suitable for use in vaccinating against infection by a mycobacterium;

a product which selectively binds a T cell receptor that recognises an epitope sequence as defined above such as the epitope sequence of formula (I), which product comprises an HLA molecule, or a fragment thereof, comprising a peptide with the sequence of the epitope sequence in its peptide binding groove;

a method of detecting in a population of T cells the presence or absence of CD8 T cells that recognise an epitope sequence as defined above such as the epitope sequence of formula (I), said method comprising: (i) contacting a population of cells comprising CD8 T cells with the polypeptide or product, and (ii) determining whether CD8 T cells recognise said polypeptide or product, to thereby determine the presence or absence of the T cells;

a method of diagnosis of a mycobacterial infection or of testing the effectiveness of a vaccination against a mycobacterial infection, said method comprising determining the presence or absence in a host of a CD8 T cell response to an epitope sequence as defined above such as the epitope sequence of formula (I), the presence of the CD8 T cell response indicating that the host has a mycobacterial infection or that the vaccination has been effective;

a T cell capable of recognising an epitope sequence as defined above such as the epitope sequence of formula (I), suitable for use in a method of treating a mycobacterial infection;

a T cell receptor, or fragment thereof, which is capable of binding an epitope sequence as defined above such as the epitope sequence of formula (I); and

a method of treating a mycobacterial infection in a pre-selected host, comprising administering to the host an effective amount of T cells as defined above.

### BRIEF DESCRIPTION OF THE DRAWINGS

FIG. 1 shows the secretion of IFN-γ by M. bovis BCG reactive CD8⁺ T cells in response to restimulation with synthetic 20-mer Ag85A peptides (overlapping by 10 aa, spanning the complete Ag85A sequence). (A) shows the mean number of sfc's (spot forming cells) per 10⁵ CD8⁺ T cells for all donors tested. (B) shows the percentage of donors who gave a positive response to each peptide, i.e. ≧50 sfc/10⁵ CD8⁺ T cells (n=12).

FIG. 2 shows IFN-γ production by PBMC stimulated overnight with either FMP M1₅₈₋₆₆, P₄₈₋₅₆, P₂₄₂₋₂₅₀, or no peptide. Results are expressed as the mean number of sfc's per 5×10⁵ PBMC.

FIG. 3 shows Ag85A peptide P₄₈₋₅₆ (A) and P₂₄₂₋₂₅₀ (B) specific cytolytic activity of short term cell lines (STCL) from 5 HLA-A*201 donors.

FIG. 4 shows CTL activity of peptide specific CD8⁺ T cells measured against autologous macrophages infected with either recombinant vaccinia virus expressing Ag85A, M. bovis BCG or M. tuberculosis.

### BRIEF DESCRIPTION OF THE SEQUENCES

SEQ ID NO: 1 shows the amino acid sequence of Ag85A₄₈₋₅₆

SEQ ID NO: 2 shows the amino acid sequence of Ag85A₂₄₂₋₂₅₀

SEQ ID NO: 3 shows the DNA and amino acid sequences of Ag 85A

SEQ ID NO: 4 shows the amino acid sequence of Ag85A

### DETAILED DESCRIPTION OF THE INVENTION

The invention is concerned with polypeptides which can consist essentially of an epitope sequence of formula (I):

X₁ L/I/M X₂ X₃ X₄ X₅ X₆ X₇ V/L/I (I)

wherein

X₁ is G, X₂ is P, X₃ is V, X₄ is E, X₅ is Y, X₆ is L and X₇ is Q, or

X₁ is K or R, X₂ is I or V, X₃ is A, X₄ is N, X₅ is N, X₆ is T and X₇ is R.

Alternatively, the polypeptides can consist essentially of an epitope sequence which is an analogue of (I) and which can be recognised by a CD8 T cell that recognises (I). The invention is also concerned with expression vectors which comprise a polynucleotide encoding such polypeptides and in which the polynucleotide is operably linked to a regulatory sequence capable of providing for expression of the polypeptide. The polypeptides and expression vectors are useful for vaccinating a host against infection by a mycobacterium. Such a vaccination may be prophylactic (of a host which does not have a mycobacterial infection) or therapeutic (of a host that does have a mycobacterial infection).

The host which is vaccinated is generally a mammal, such as a human or animal, typically one which can be naturally or artificially infected by a mycobacterium. The host may be a primate, cow or badger. The host may be at risk of a mycobacterial infection, typically because it is resident in a location in which mycobacterial infection is endemic. The host may be susceptible to mycobacterial infection due to malnutrition or infection by other pathogens, such as HIV.

The mycobacterium against which the host is vaccinated expresses the Ag85A protein, or a protein which comprises the epitope sequence (such as a homologue of the Ag85A sequence shown as SEQ ID NO: 4). The mycobacterium is typically pathogenic and capable of infecting mammals, such as those mammals discussed above. The mycobacterium is typically M. tuberculosis, M. marinum, M. kansasii, M. bovis or M. avium.

The vaccination stimulates a CD8 T cell response to the epitope (which may comprise responses to different epitopes of the invention as defined by (I) or the analogue of (I)). The vaccination may or may not lead to a CD8 T cell response to one or more other epitopes in Ag85A, or to one or more epitopes in other mycobacterial proteins. The vaccination may or may not lead to an antibody response that recognises an epitope in Ag85A or in 1, 2, 3 or more other mycobacterial proteins.

The epitope sequence may have the sequence of formula (I). Preferably the epitope has the sequence GLPVEYLQV (SEQ ID NO: 1) or KLIANNTRV (SEQ ID NO: 2). The epitope may have a sequence which is an analogue of (I) (including analogues of SEQ ID NO: 1 or 2) which is recognised by a T cell that recognises (I). Such T cell recognition includes the binding of the T cell receptor to the analogue, and typically also antigen-specific functional activation of the T cell.

Thus typically a peptide with the sequence of the analogue (referred to as the analogue peptide herein) is capable of inhibiting the binding of a peptide of sequence (I) (referred to as peptide (I) herein) to a T cell receptor. Generally therefore the amount of peptide (I) which can bind the T cell receptor in the presence of the analogue peptide is decreased. This is because the analogue peptide is able to bind the T cell receptor and therefore competes with the epitope for binding to the T cell receptor. The binding of the analogue peptide to the T cell receptor is a specific binding. Generally during the binding discussed above peptide (I) and the analogue peptide are bound to an MHC class I molecule, such as HLA-A*0201.

The inhibition of binding can be determined using techniques known in the art or any of the techniques or under any of the conditions discussed herein. The T cell receptor used binds specifically to the peptide (I). T cells with such receptors can be produced by stimulating antigen naive T cells in vitro or in vivo with peptide (I), which is generally presented to the T cell by an appropriate HLA molecule.

Antigen-specific functional activation of the T cell by the analogue peptide may be measured using suitable technique discussed herein. Generally the analogue peptide causes such activation when it is presented to the T cell associated with an MHC class I molecule, such as HLA-A*0201 (for example on the surface of a cell).

The analogue peptide (or analogue sequence within a larger peptide) is typically capable of stimulating a CD8 T cell response directed to peptide (I), for example when administered to a human or animal (such as in any of the forms or with any of the adjuvants mentioned herein). Such a response may be protective against tuberculosis in an animal model or of therapeutic benefit in a human patient.

The analogue peptide typically has a shape, size, flexibility or electronic configuration which is substantially similar to peptide (I). It is typically a derivative of the peptide (I).

As well as binding the T cell receptor as discussed above, the analogue peptide may also be able to bind other specific binding agents that bind the peptide (I). Such an agent may be HLA-A*0201. The analogue peptide typically binds to antibodies specific for peptide (I), and thus inhibits binding of peptide (I) to such an antibody. The analogue peptide is either a peptide or non-peptide or may comprise both peptide and non-peptide portions. Such a peptide or peptide portion may have homology with the peptide (I) (e.g. homology with SEQ ID NO: 1 or 2).

The analogue sequence may comprise 1, 2, 3, 4 or more non-natural amino acids, for example amino acids with a side chain different from natural amino acids. Generally, the non-natural amino acid will have an N terminus and/or a C terminus. The non-natural amino acid may be an L- or a D-amino acid.

Typically the analogue sequence is a peptide sequence which comprises one or more modifications. The modification may be any of those mentioned herein which can be present on the polypeptide of the invention. The modification can be present on any of the amino acids of the analogue sequence, such as any of the amino acids responsible for binding the MHC molecule or which contact the T cell receptor during recognition by a T cell.

The analogue sequence is typically designed or selected by computational means and then synthesised using methods known in the art. Alternatively the analogue can be selected from a library of compounds. The library from which the analogue sequence is selected is typically a library comprising peptides, such as peptides which have an HLA-A*0201 binding motif.

The library may be a combinatorial library or a microorganism display library, such as a phage display library. The library of compounds may be expressed in the display library in the form of being bound to a MHC class I molecule, such as HLA-A*0201.

An analogue peptide or sequence can be selected from the library based on any of the characteristics mentioned above, such as the ability to mimic the binding characteristics of peptide (I), for example the ability to bind a T cell receptor, HLAA*-0201 or antibody which recognises peptide (I). The analogue may be selected based on the ability to cause antigen specific functional activity of a T cell that recognises peptide (I) (for example using any of the suitable techniques mentioned herein).

The polypeptide is generally 8 to 2000 amino acids in length, such as 9 to 1000, 10 to 500, 11 to 200, 12 to 100 or 15 to 50 amino acids in length. Typically the polypeptide has a length of up to 50 amino acids. The polypeptide is typically a non-naturally occurring protein, such as a fusion protein comprising sequence from the same or different proteins. A preferred fusion protein comprises the derivative the sequence of Ag85A discussed below.

The polypeptide may comprise a sequence which is a derivative of the sequence of Ag85A (e.g. as shown in SEQ ID NO: 3). In the case where the polypeptide comprises sequence other than the derivative sequence such sequence is typically not a derivative of the sequence of Ag85A (derivative being defined according to any manner mentioned herein). The derivative sequence will typically comprise the epitope sequence. Such a derivative may be fragment of Ag85A. In one embodiment the fragment only contains Ag85A sequence that lies close to the epitope sequence, such as only sequence consisting of or within positions 43 to 61, 38 to 66, 28 to 76, 236 to 254,231 to 259 or 221 to 269. In one embodiment the fragment only comprises the epitope sequence.

The derivative of the Ag85A sequence may be homologous to the whole Ag85A sequence or any of the fragments thereof mentioned above.

The polypeptide typically comprises 1, 2, 3, 4, or from 5 to 10, or more copies of the epitope sequence, which may be the same or different. Typically in the polypeptide, a linker sequence may or may not separate the epitope sequences and/or there may or may not be additional (non-epitope) sequence at the N terminal or C terminal of the peptide. Typically the peptide comprises 1, 2, 3 or more linkers. The linkers are typically 1, 2, 3, 4 or more amino acids in length. Thus in the peptide 1, 2 or more, or all of the epitope sequences may be contiguous with each other or separated from each other.

The polypeptide may also comprise sequence which enhances the immunogenicity of the epitope sequence. Such sequences are discussed below.

The polypeptide may also comprise 1, 2, 3, 4 or from 5 to 10, or more, other epitope sequences, such as other CD8 T cell epitope sequences (which are recognised by different T cells), CD4 T cell epitopes or antibody epitopes. When the polypeptide or expression vector is administered to the host an immune response may also generated against these epitopes. The other epitope(s) may be mycobacterial epitopes (such as epitopes from mycobacterial proteins other than Ag85A). The other epitope(s) may be of another pathogen, such as a viral pathogen (e.g. HIV). The other epitope(s) may be of Clostridium (e.g. from the Clostridium tetani neurotoxin fragment C), hepatitis B (e.g. from core or surface protein). The other epitope(s) may be artificial or consensus epitope (such as PADRE), for example as described in del Guercio et al (1997) Proc. Natl. Acad. Sci. USA 93, 11786-91.

In one embodiment the polypeptide is modified, for example by a natural post-translational modification (e.g. glycosylation) or an artificial modification. The peptide may comprise the modifications that occur when it is expressed in a eukaryotic (e.g. human) or prokaryotic (e.g. E. coli) cell. In one embodiment the peptide lacks glycosylation. The modification may provide a chemical moiety (typically by substitution of a hydrogen, e.g. the hydrogen of a C—H bond), such as an amino, acetyl, hydroxy or halogen (e.g. fluorine) group or carbohydrate group. Typically the modification is present on the N or C terminus.

The polypeptide is typically capable of being processed by the class I antigen presenting pathway of a cell to provide a peptide (peptide (I) or the analogue peptide) on the surface of the cell bound a MHC class I molecule. Typically such a cell is able to present the peptide to a T cell.

The polypeptide may be produced synthetically or expressed in a recombinant system. Solid phase or solution phase synthesis methods may be used. In solid phase synthesis the amino acid sequence of the desired peptide is built up sequentially from the C terminal amino acid which is bound to an insoluble resin. When the desired peptide has been produced it is cleaved from the resin. In solution phase synthesis the desired peptide is again built up from the C terminal amino acid. The carboxy group of this amino acid remains blocked throughout by a suitable protecting group, which is removed at the end of the synthesis.

In both solid phase and solution phase synthesis each amino acid added to the reaction system typically has a protected amino group and an activated carboxy group. Functional side chains are also protected. After each step in the synthesis the amino-protecting group is removed. Side chain functional groups are generally removed at the end of the synthesis.

The expression vector capable of expressing the polypeptide is a polynucleotide, typically DNA or RNA, and is typically single or double stranded. The polynucleotide may be linear or circular (e.g. a plasmid). Generally when the expression vector is RNA it is capable of being directly translated to provide the polypeptide (and typically does not contain the sequences mentioned below which aid transcription). The vector comprises a sequence which encodes the polypeptide, which is typically operably linked to a control sequence capable of providing for expression of the coding sequence. Thus typically the vector comprises 5′ and 3′ to the coding sequence sequences which aid expression, such as aiding transcription and/or translation of the coding sequence. Typically the vector comprises a promoter, enhancer, transcription terminator, polyadenylation signal, polyA tail, intron, translation initiation codon or translation stop codon.

The expression vector is capable of expressing the polypeptide. In one embodiment the vector is capable of expressing the polypeptide in a cell of the host (using the host cell transcription and translation mechanisms). In another embodiment the expression vector is within a cellular vector (as discussed below), and therefore is capable of expressing the polypeptide in the cellular vector (using the transcription and translation mechanisms of the cellular vector).

The vector may also be capable of expressing a substance which enhances the immunogenicity of the polypeptide (such as any such substance mentioned herein). In one embodiment the vector comprises 2 or more coding sequences, and may be capable of expressing 2 or more different polypeptides of the invention.

The vector may be associated with (e.g. within) a moiety which aids delivery or expression of the vector. Preferably such a moiety aids delivery of the polypeptide expressed by the vector to the class I processing and presentation pathway. Such a moiety may be a virus or cell. Thus in one embodiment the vector is present in a virus vector (i.e. a viral vector), such as a virus which is capable of stimulating a CD8 T cell response (e.g. a vaccinia virus). The virus is typically one whose wild-type is capable of infecting the host. The cell in which the vector may be present may be a prokaryotic (e.g. bacterium) or eukaryotic cell. It is typically the cell of a pathogen which is capable of infecting the host. Such a pathogen may be virulent in its wild-type form. In one embodiment the cell is of a mycobacterium (such as any of those mentioned herein including M. bovis bacillus Calmette-Guerin). The cell may be of the species of the host or may be a cell which has previously been extracted from the host (and optionally cultured and/or replicated in vitro). Such a cell may be any type of cell mentioned herein, and is typically a professional antigen presenting cell.

A recombinant replicable polynucleotide vector which comprises a sequence that encodes the polypeptide of the invention may also be mentioned. In one embodiment this is the same as the expression vector of the invention. The vector may be replicated in a compatible cell. Thus in a further embodiment, the invention provides a method of making vectors of the invention by introducing a polynucleotide sequence that encodes the polypeptide of the invention into a replicable vector, introducing the vector into a compatible cell, and growing the cell under conditions which bring about replication of the vector. The vector may be recovered from the cell. Suitable cells are described below.

Preferably, the sequence which encodes the polypeptide is operably linked to a control sequence which is capable of providing for the expression of the coding sequence by the cell.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Such vectors may be transformed into a suitable cell to provide for expression of a polypeptide of the invention. Thus, a polypeptide according to the invention can be obtained by cultivating a cell transformed or transfected with a vector as described above under conditions to provide for expression of the polypeptide, and recovering the expressed polypeptide.

The vector may be for example, plasmid, virus or phage vector provided with an origin of replication, optionally a promoter for the expression of the coding sequence and optionally a regulator of the promoter. The vector may contain one or more selectable marker genes, for example an ampicillin resistance gene in the case of a bacterial plasmid. Promoters and other expression regulation signals may be selected to be compatible with the cell for which the vector is designed.

Cells transformed (or transfected) with the vector will be chosen to be compatible with the said vector and preferably will be bacterial such as E. coli. Alternatively they may be cells of a human or animal cell line such as CHO or COS cells, or yeast or insect -cells. The cells may also be cells of a non-human animal such as a sheep or rabbit or plant cells.

As mentioned above the polypeptide may comprise a sequence which enhances the immunogenicity of the epitope. The polypeptide or expression vector may also be associated with a substance (which may be a peptide or non-peptide substance) or may be in a form which is capable of enhancing the immunogenicity of the epitope. Generally this will enhance the speed and/or magnitude of the CD8 T cell response to the epitope, and thus generally after vaccination a larger number of CD8 T cells specific for the epitope will be present in the host (for example in the peripheral blood).

The substance (including the sequence which may be present in the polypeptide) may act as adjuvant or may target the peptide to antigen presenting cells (APCs) or to compartments in the antigen processing pathway, for example acting as a carrier protein. The sequence may stimulate a T helper response, such as a response that favours a CD8 T cell response, and thus may comprise a T helper (e.g. Th1) cell epitope.

Preferred substances which enhance immunogenicity include sequence from the hepatitis B core antigen, sequence from a stress protein or sequence from Clostridium tetani neurotoxin fragment C. The stress protein is typically a bacterial (e.g. mycobacterial) heat shock protein (HSP) or a protein which has homology with such a protein, such as mycobacterial or E. coli proteins of the HSP 60 and HSP 70 families (e.g. HSP 65 or HSP 71 of mycobacteria) or mammalian homologue (e.g. gp96 of mice or humans, Anthony et al (1999) Vaccine 17, 373-83).

The substance may cause the polypeptide or vector to adopt a particulate form. The substance may be a virus or virus-like particle (such as a yeast Ty particle, e.g. as in Allsopp et al (1996) Eur. J. Immunol. 26, 1951-9). The substance may be a cytokine, such as a cytokine which stimulates a MHC class I restricted T cell response or favourable MHC class II restricted T cell response (e.g. IL-2, IL-7, IL-12, IFN-γ or GMCSF). The substance may be, for example, CFA, a muramyl dipeptide (e.g. of a mycobacterial cell wall), monophosphoryl lipid A, lipopolysaccharide (e.g. from B. abortus), liposomes, SAF-1, a saponin (e.g. Quil A), keyhole limpet hemocyanin, beta 2-microglobulin, mannan (e.g. oxidised mannan), an acrylic based microbead, or an emulsion (e.g. oil in water or water in oil) such as soybean emulsion (e.g as in Hioe et al (1996) Vaccine 14, 412-8).

The particular route of administration used may aid the stimulating of a CD8 T cell response, and thus the polypeptide vector may be provided in a form suitable for administering by such a route. Delivery by an intramuscular route or by biolistic means is preferred.

Generally a low dose of antigen favours the development of a CD8 T cell response. Thus a suitable low dose of the polypeptide or vector may be administered. The polypeptide or vector may thus be in an amount and concentration that is suitable for administering to provide an appropriate low dose. In a preferred embodiment the vector is administered in the form of “naked DNA”.

The product of the invention selectively binds a T cell receptor of the T cell of the invention, typically in a reversible manner. Such a product is generally able to inhibit the binding of epitope peptide (e.g. bound to an MHC molecule) to the T cell receptor.

As mentioned above the product comprises an HLA molecule, or a fragment thereof, comprising a peptide with the sequence of the epitope sequence in its peptide binding groove. The HLA molecule is generally a MHC class I molecule (e.g. HLA-A*0201). Such molecules comprise an a chain and a β chain. The fragment may comprise only the extracellular domain of the HLA molecule. The fragment may or may not be capable of binding a cell membrane. In one embodiment the HLA molecule an a chain and a β chain which are not naturally occurring chains, but will typically have homology with naturally occurring chains (such as those of HLA-A*0201).

In one embodiment 2, 3, 4 or more products are linked together in a multimer. The products in the multimer may be linked by a covalent bond or by non-covalent means. In a preferred embodiment the products are linked by a streptavidin-biotin interaction, and thus typically the products comprise a biotin portion.(typically chemically linked to or in a fusion protein with the product) which allows the products to be linked together by streptavidin.

The multimer generally has a-higher binding affinity to the T cell receptor of the invention than the product. The multimer may also comprise a detectable label, such as a radioactive or a light detectable (e.g. fluorescent) label. The label may allow the multimer to be sorted by flow cytometry-(e.g. when the multimer is bound to a T cell receptor which is present on a T cell of the invention).

The multimer may be a soluble multimer or may be capable of associating with a cell membrane. In one embodiment the multimer is attached to a solid support, such as a microtitre plate.

As mentioned above, the invention provides a method of detecting the presence or absence of CD8 T cells that recognise the epitope sequence (which may be in the form of the product discussed above). In one embodiment the determination of whether the T cells recognise the epitope sequence is done by detecting a change in the state of the T cells in the presence of the epitope sequence or determining whether the T cells bind the epitope sequence. The change in state is generally caused by antigen specific functional activity of the T cell after the T cell receptor binds the epitope sequence. Generally when binding the T cell receptor the epitope sequence is bound to an MHC class I molecule, which is typically present on the surface of an APC (antigen presenting cell) and typically presented by an appropriate HLA molecule.

The change in state of the T cell may be the start of or increase in the expression of a substance in the T cells and/or secretion of a substance from the T cell, such as a cytokine (e.g. IFN-γ, IL-2 or TNF-α). Determination of IFN-γ expression or secretion is particularly preferred. The substance can typically be detected by allowing it to bind to a specific binding agent and then measuring the presence of the specific binding agent/substance complex. The specific binding agent is typically an antibody, such as polyclonal or monoclonal antibodies. Antibodies to cytokines are commercially available, or can be made using standard techniques.

Typically the specific binding agent is immobilised on a solid support (and thus the method may based on the ELISPOT assay to detect secretion of the substance). After the substance is allowed to bind the solid support can optionally be washed to remove material which is not specifically bound to the agent. The agent/substance complex may be detected by using a second binding agent which will bind the complex. Typically the second agent binds the substance at a site which is different from the site which binds the first agent. The second agent is preferably an antibody and is labelled directly or indirectly by a detectable label.

Thus the second agent may be detected by a third agent which is typically labelled directly or indirectly by a detectable label. For example the second agent may comprise a biotin moiety, allowing detection by a third agent which comprises a streptavidin moiety and typically alkaline phosphatase as a detectable label.

Alternatively the change in state of the T cell which can be measured may be the increase in the uptake of substances by the T cell, such as the uptake of thymidine. The change in state may be an increase in the size of the T cells, or proliferation of the T cells, or a change in cell surface markers on the T cell.

The change in state may be the killing (by the T cell) of a cell which presents the epitope sequence. Thus the determination of whether the T cells recognise the peptide may be carried out using a CTL assay.

In one embodiment the T cells which are contacted in the method are taken from the host in a blood sample, although other types of samples which contain T cells can be used. The sample may be added directly to the assay or may be processed first. Typically the processing may comprise diluting of the sample, for example with culture medium or buffer. Typically the sample is diluted from 1.5 to 100 fold, for example 2 to 50 or 5 to 10 fold.

The processing may comprise separation of components of the sample. Typically mononuclear cells (MCs) are separated from the sample. The MCs will include the T cells and APCs. Thus in the method the APCs present in the separated MCs can present the peptide to the T cells. In another embodiment only T cells, (in one embodiment only CD8 T cells), can be purified from the sample. PBMCs (peripheral blood mononuclear cells), MCs and T cells can be separated from the sample using techniques known in the art.

The T cells used in the assay can be in the form of unprocessed or diluted samples, or are freshly isolated T cells (such as in the form of freshly isolated MCs or PBMCs) which are used directly ex vivo, i.e. they are not cultured before being used in the method. However, more typically the T cells are cultured before use, for example in the presence of the epitope sequence, and generally also exogenous growth promoting cytokines. During culturing the epitope sequence is typically present on the surface of APCs, such as the APC used in the method. Pre-culturing of the T cells may lead to an increase in the sensitivity of the method.

The APC which is typically used in the method is from the-same host as the T cell or from a different host. The APC can be an non-professional APC, but is typically a professional APC, such as any of the APCs mentioned herein. The APC maybe an artificial APC. The APC is a cell which is capable of presenting the peptide to a T cell. It is typically separated from the same sample as the T cell and is typically co-purified with the T cell. Thus the APC may be present in MCs or PBMCs. The APC is typically a freshly isolated ex vivo cell or a cultured cell. It may be in the form of a cell line, such as a short term or immortalised cell line. The APC may express empty MHC class I molecules on its surface.

In one embodiment the polypeptide per se is added directly to an assay comprising T cells and APCs. This may be taken up directly onto the surface of the APCs (particularly if the APCs express empty MHC class I molecules on their surface). In one embodiment the polypeptide is provided to the APC in the absence of the T cell. The APC is then provided to the T cell, typically after being allowed to present the epitope sequence on its surface.

The duration for which the epitope sequence is contacted with the T cells will vary depending on the method used for determining recognition of the peptide. Typically the concentration of T cells used is 10³/ml to 10⁹/ml, preferably 10⁵/ml to 10⁷/ml. In the case where polypeptide is added directly to the assay its concentration is typically from 0.1 to 1000 μg/ml, preferably 10 to 100 μg/ml. Typically the length of time for which the T cells are incubated in the assay is from 4 to 24 hours, preferably 6 to 16 hours, and in one embodiment for at least 40, 120 or 180 hours.

The determination of the recognition of the epitope sequence by the T cells may be done by measuring the binding of the epitope sequence to the T cells. Typically T cells which bind the peptide can be counted and sorted based on this binding, for example using a FACS machine. The presence of T cells which recognise the peptide will be deemed to occur if the frequency of cells identified using the peptide is above a ‘control’ value (i.e. above the frequency of antigen naive T cells which recognise the epitope sequence). The frequency of antigen-experienced T cells specific for a particular epitope during a disease state would be expected to be between 5 and 60% of the total CD8 T cells.

As mentioned above the invention provides a method of diagnosis of a plycobacterial infection or of testing the effectiveness of a vaccination against a mycobacterial infection. It may not be possible to distinguish between the host having a CD8 T cell response to the epitope due to mycobacterial infection or due to vaccination. A host which is found not to have a CD8 T cell response (or a level of CD8 T cell response which is not sufficient to provide protective/sterilising immunity) to the epitope may be selected for vaccination to stimulate such a T cell response (e.g. by the vaccination method mentioned herein).

In the method of diagnosis or testing the effectiveness of a vaccination of the invention the presence or absence of the CD8 T cell response is typically determined by the method of identifying a CD8 T cell response discussed above.

The invention also provides a CD8 T cell that recognises the epitope sequence, i.e. the TCR of the T cell is able to bind a peptide with the epitope sequence (typically when the peptide is bound to an appropriate HLA molecule and presented on a cell). Generally the T cell is able to undergo antigen specific functional activation upon binding the epitope sequence. The T cell typically produces IFN-γ, and typically does not produce IL-4, IL-10 or TGF-β. The T cell may be cytotoxic and typically contains cytotoxic granules comprising perforin, granzymes and granulysin. Preferably the T cell is cytotoxic towards autologous macrophages infected with a mycobacterium (such as any mycobacterium mentioned herein). Generally the T cell is not immunosuppressive.

Typically the T cell has previously been extracted from the same host and has been replicated in vitro. Such replication may comprise culturing in the presence of the epitope sequence (typically presented on the surface of a cell). The cell may be used in a method of treating a mycobacterial infection.

The invention provides a T cell receptor (TCR), or fragment thereof, which is capable of binding the epitope sequence. The TCR may have any of the properties of the TCR present on the T cell of the invention. Thus the epitope sequence is generally bound to an MHC molecule during binding with the TCR.

The polypeptide, expression vector, cell (including T cell), product and TCR (or fragment thereof) of the invention may be in substantially purified form. They may be in substantially isolated form, in which case they will generally comprise at least 80% e.g. at least 90, 95, 97 or 99% of the polypeptide, polynucleotide, cells or dry mass in the preparation. The polypeptide, expression vector, product or TCR (or fragment thereof) is typically substantially free of other cellular components, or free of other mycobacterial components, or free of (other) polypeptide or polynucleotide. The polypeptide, expression vector, cell or product may be used in any of the above forms in any aspect of the invention mentioned herein.

The polypeptide and expression vector discussed above in any form or in association with any other agent is included in the termed “vaccination agent” below. An effective non-toxic amount of such a vaccination agent may be given to a pre-selected host, such as a human or non-human. The vaccination agent may be administered prophylactically to a host who does not have a mycobacterial infection in order to prevent the individual developing a mycobacterial infection. The vaccination agent may be administered therapeutically to a host who has a mycobacterial infection, in order to treat the infection (including ameliorating the symptoms of the disease). The T cells of the invention may be administered in an effective non-toxic amount to a patient in need thereof, such as a patient with a mycobacterial infection.

Thus the invention provides the vaccination agent and the T cells for use in a method of treating the human or animal body by therapy, and for use in the manufacture of a medicament for the treatment of a mycobacterial infection.

The vaccination agent or the T cells may be given to the host in one or more administrations. Typically after the initial administration a ‘booster’ can be given. Typically the host is given 1, 2, 3 or more separate administrations, each of which is separated by at least 12 hours, 1 day, 2, days, 7 days, 14 days, 1 month or more.

The vaccination agent or T cells may be in the form of a pharmaceutical composition which comprises the vaccination agent or T cells and a pharmaceutically acceptable carrier or diluent. Suitable carriers and diluents include isotonic saline solutions, for example phosphate-buffered saline. Typically the administration is (and thus the composition is formulated for) parenteral, intravenous, intramuscular, subcutaneous, transdermal, intradermal, oral, intranasal, intravaginal, or intrarectal administration.

A preferred method of delivering the vaccination agent (particularly in the case of the expression vector) is by biolistic means (a particle-mediated method). In such a technique the agent is typically coated onto carrier particles using a variety of techniques known in the art. Carrier particles are selected from materials which have a suitable density in the range of particle sizes typically used for (e.g. intracellular) delivery from a gene gun device. The optimum carrier particle size will, of course, depend on the desired target (e.g. diameter of the target cells).

For the purposes of the invention, tungsten, gold, platinum and iridium carrier particles can be used. Tungsten and gold particles are preferred. Tungsten particles are readily available in average sizes of 0.5 to 2.0 μm in diameter. Such particles have optimal density for use in particle acceleration delivery methods, and allow highly efficient coating with DNA. Gold particles or microcrystalline gold (e.g., gold powder A1570, available from Engelhard Corp., East Newark, N.J.) provide uniformity in size (available from Alpha Chemicals in particle sizes of 1-3 μm, or available from Degussa, South Plainfield, N.J. in a range of particle sizes including 0.95 μm) and may also be less toxic than tungsten. Microcrystalline gold provides a diverse particle size distribution, typically in the range of 0.5-5 μm.

A number of methods are known and have been described for coating or precipitating agents onto particles (e.g. combining a predetermined amount of gold or tungsten with plasmid DNA, CaCl₂ and spermidine). The resulting solution is vortexed continually during the coating procedure to ensure uniformity of the reaction mixture. After precipitation of the agent, the coated particles can be transferred to suitable membranes and allowed to dry prior to use, coated onto surfaces of a sample module or cassette, or loaded into a delivery cassette for use in particular gene gun instruments.

Following their formation, particles coated with the agent are delivered using particle-mediated delivery techniques. Suitable particle acceleration devices are known in the art. Typically such a device employs an explosive, electric or gaseous discharge to propel coated particles towards the target. The coated particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is the ACCELL® instrument (Geniva, Madison, Wis.), which instrument is described in U.S. Pat. No. 5,120,657. Another electric discharge apparatus suitable for use herein is described in U.S. Pat. No. 5,149,655.

A single administration may include more than one gene gun dose, e.g. two to six gene gun doses.

The dose of vaccination agent administered (by a method) may be determined according to various parameters, especially according to the substance used; the age, weight and condition of the patient to be treated; the route of administration; and the required regimen. A physician will be able to determine the required route of administration and dosage for any particular patient. A suitable dose may however be from 10 μg to log, for example from 100 μg to 1 g of the vaccination agent or T cells. These values may represent the total amount administered in the complete treatment regimen or may represent each separate administration in the regimen.

The polypeptides of the invention may be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

In the case of the expression vector transfection agents may also be administered to enhance the uptake of the expression vector by cells. Examples of suitable transfection agents include cationic agents (for example calcium phosphate and DEAE-dextran) and lipofectants (for example lipofectam™ and transfectam™).

When the vaccination agent is in the form of a viral vector the amount of virus administered is in the range of from 10⁴ to 10¹² pfu, preferably from 10⁷ to 10¹⁰ pfu (for example for adenoviral vectors), more preferably about 10⁸ pfu for herpes viral vectors. A pox virus vector may also be used (e.g. vaccinia virus), typically at any of the above dosages. When injected, typically 1-2 ml of virus in a pharmaceutically acceptable suitable carrier or diluent is administered. When cells are administered typically 10⁵ to 10¹³ cells, preferably 10⁸ to 10¹¹ cells are administered.

Homologous Sequences

Homologous sequences (of polypeptides or within polypeptides) are mentioned herein. Such sequences typically have at least 70% homology, preferably at least 80, 90%, 95%, 97% or 99% homology with the relevant sequence, for example over a region of at least 15, 20, 40, 100 more contiguous amino acids (of the homologous sequence). Methods of measuring protein homology are well known in the art and it will be understood by those of skill in the art that in the present context, homology is calculated on the basis of amino acid identity (sometimes referred to as “hard homology”).

For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment, The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologous sequence typically differs from the relevant sequence by at least (or by no more than) 2, 5, 10, 15, 20 more mutations (which may be substitutions, deletions or insertions). These mutations may be measured across any of the regions mentioned above in relation to calculating homology. The substitutions are preferably “conservative”. These are defined according to the following Table. Amino acids in the same block in the second column and preferably in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

In the case of the analogue sequence this typically differs from the epitope sequence (such as SEQ ID NO: 1 or 2) by at least (or no more than) 1, 2, 3, 4 or more mutations (which may be insertions, deletion or substitution (e.g. conservative substitutions)).

Homologous sequences mentioned herein may be encoded by a polynucleotide which hybridises to a polynucleotide that encodes the relevant polypeptide, typically hybridising selectively at a level significantly above background. Selective hybridisation is typically achieved using conditions of medium to high stringency (for example 0.03M sodium chloride and 0.003M sodium citrate at from about 50° C. to about 60° C.). However, such hybridisation may be carried out under any suitable conditions known in the art (see Sambrook et al (1989), Molecular Cloning: A Laboratory Manual). For example, if high stringency is required, suitable conditions include 0.2×SSC at 60° C. If lower stringency is required, suitable conditions include 2×SSC at 60° C.

The following Examples illustrate the invention.

### EXAMPLE 1

Techniques

M. bovis BCG and M. tuberculosis Culture

M. bovis BCG (Glaxo Evans strain; Evans Medical, Leatherhead, UK) and M. tuberculosis (H37Rv) were grown to log phase in Middlebrookes 7H9 medium (Detroit, Mich., USA) supplemented with Middlebrookes ADC enrichment (Difco) and 0.05% Bacto-Glycerol (Difco). Bacteria were harvested and frozen at −70° C. Bacterial counts were determined by counting CFUs either grown on Middlebrooks 7H10 agar supplemented with OADC enrichment (Difco) or in liquid 7H9 agar supplemented with ADC enrichment (Difco). Vials were thawed and washed once with RPMI-1640 (Gibco-BRL, Paisely, UK) and sonicated for 10 seconds in a water bath sonicator (Grant XB2; Fisions, Leicester, UK) to disrupt clumps before use.

Recombinant Vaccinia Virus Construction

Recombinant vaccinia virus expressing the Ag85A protein from M. tuberculosis was Harboe et al (1998) Infect. Immun. 66, 717-23. The coding sequences of the Ag85A protein were cloned into the nonessential thymidine kinase locus of wild type vaccinia virus using the transfer plasmid p1108; a negative control recombinant vaccinia virus was constructed by using the p1108 plasmid with an irrelevant coding sequence. Recombinant vaccinia viruses were produced by transfection of the plasmid into the thymidine kinase disrupted osteosarcoma cell line 143 (TK′143) coinfected with wild-type vaccinia virus followed by selection for recombinant viruses. The resulting recombinant viruses were plaque purified and protein expression was confirmed by PCR and Western blot analysis of infected TK′ 143 cells.

THP-1 Cell Culture

The human monocyte cell line THP-1 was maintained in T75 tissue culture flasks Nunclon, Roskilde, Denmark). The growth medium used was RPMI-1640 supplemented with 2 mM L-glutamine (Gibco-BRL) and 10% heat-inactivated FCS (Gibco-BRL).

PBAMC Separation

PBMC were isolated from heparanised venous blood by density gradient centrifugation over Ficoll-Histopaque (Sigma Chemical Co., Poole, Dorset, UK). Non-adherent cells (NAC) and monocytes were separated by adherence to plastic. PBMC were resuspended at 1×10⁶ cells/ml in RPMI-1 640 and plated at 200 μl/well of a 96 well round bottomed tissue culture plate. Cells were incubated for 1 hour at 37° C. before NAC were removed by pipetting off the supernatant. Adherent monocytes were then resuspended in culture medium (CM) and incubated at 37° C., 5% CO₂ for use as target cells in CTL assays. CM consisted of RPMI-1640 supplemented with 2 mM L-glutanine (Gibco-BRL), 50 μg/ml ampicillin (Sigma) and 10% autologous plasma. NAC were used for the generation of peptide specific short-term cell lines (STCL).

Generation of CD8⁺ T Cell Lines

NAC alone were prepulsed with 50 μM peptide for 1 hour in a cell pellet and then diluted up to 1×10⁶ cells/ml in CM supplemented with 25 ng/ml ryIL-7 (R & D Systems, Abingdon, UK) and seeded at 100 μl/well in a 96 well round bottomed tissue culture plate. Culture medium containing 10% Lymphocult-T (Biotest, Soilhull, UK) was added to each well at 3-4 day intervals. For the generation of M. bovis BCG cell lines, PBMC were aliquoted at 100 μl/well and infected with 2×10⁴ bacilli per well. Cells were cultured as for peptide specific cell lines.

CD8⁺ T Cell Separation

Antigen-specific CD8+T cells were prepared by positive enrichment using the MACS system (Miltenyi Biotech, Bergisch-Gladbach, Germany). In brief, PBMC were labelled with CD8 microbeads (Miltenyi Biotech; 20 μl/10⁷ cells) in incubation buffer (PBS/0.5% BSA/2 mM EDTA; 80 μl/10⁷ cells) for 15 minutes at 4° C. After one washing cells were resuspended in incubation buffer (1 ml buffer/10⁷ cells) and enrichment was performed using LS⁺ columns and the MidiMACS magnet according to manufacturer's instructions. The resulting CD8⁺ T-cell population was >95% pure as determined by flow cytometric analysis for the surface markers αβTCR, CD3, CD8; and contamination with CD4 and CD56.

ELISPOT Assay for IFN-γ

Ninety-six-well polyvinylidene difluoride (PVDF)-backed plates (MAIP S 45; Millipore, Bedford, UK) were precoated with 15 μg/ml anti-IFN-γ mAb (1-D1K; Mabtech, Stockholm, Sweden) overnight at 4° C. Plates were then washed 6 times with RPMI-1640 and blocked with RPMI-1640 supplemented with 10% heat-inactivated FCS for 1 hour at 37.

Purified CD8⁺ T cells from short term cell lines were washed twice with RPMI-1640, resuspended in RPMI-1640 supplemented with L-glutamine and 10% autologous plasma, and dispensed at known input cell numbers per well in duplicate wells. Peptide was added directly to the supernatant at a final concentration of 2 μM and the plates incubated at 37° C. for 18 hours. Cells were then shaken off and the plate washed six times with PBS 0.05% Tween-20 (Sigma). Plates were then incubated for 3 hours at 37° C. with the second biotinylated anti-IFN-γ mAb (7-B6-1 biotin at 1 μg/ml; Mabtech). A further wash with PBS 0.05% Tween-20 was performed before the addition of a 1:1000 dilution of strepavadin-alkaline phosphatase conjugate (Mabtech) for 2 hours.

Wells were again washed six times and 100 μl of chromogenic alkaline phosphatase substrate (Bio-Rad, Hemel Hempstead, Hertfordshire, UK) was added to each well. After 30 minutes, the colormetric reaction was terminated by washing with tap water and the plates were allowed to air dry. After drying spots were counted.

⁵¹Cr Release Cytotoxicity Assays

Autologous monocyte-derived-macrophages or the cell line THP-1 were seeded at 10,000 cells/well of a 96 well round bottomed plate and used as target cells in cytotoxicity assays. Target cells were incubated overnight at 37° C. in CM (RPMI supplemented with L-glutamine plus 10% autologous plasma for macrophages or 10% heat inactivated pooled human AB serum for THP-1 cell line), loaded with antigen (10 μg/ml peptide; 10:1 (CFU/macrophage) M. bovis. BCG or M. tuberculosis; 25:1 (pfu/macrophage) rVV expressing Ag85A; or no antigen) and pulsed with 2 μCi ⁵¹Cr.

Target cells were washed three times with RPMI and either treated with 5 μg/ml w6/32 mAb (Sigma) or resuspended in CM alone before being used in the cytotoxicity assay. Purified CD8⁺ T cells were added as effector cells to the 96 well plate at various E:T ratios (50:1, 5:1, 0:1) in a final volume of 100 μl/well and incubated at 37° C. for 6 hours. Supernatants (50 μl) were harvested, and ⁵¹Cr release was measured using a gamma counter. The remaining supernatant was removed and replaced with 100 μl/well 5% SDS (Sigma) for 1 hour at 37° C. to lyse the remaining cells.

Spontaneous release was measured in wells containing target cells alone. The percent specific lysis was calculated by the following formulae:

$$\begin{matrix}\% Isotope release = cpm supernatant / ( cpm supernatant + cpm pellet ) ] \times 100 \\ \% Specific lysis = \% isotope release test wells - \% isotope release control wells . \end{matrix}$$

### EXAMPLE 2

Identification of CD8⁺ T Cell Reactive Peptides

PBMC from 10 healthy donors who had previously received M. bovis bacillus Calmette-Guerin (BCG) vaccination were stimulated for 14 days with live M. bovis BCG (Glaxo Evans Strain; Evans Medical, Leatherhead, UK) in the presence of IL-2 and IL-7 to produce BCG reactive STCL. CD8⁺ T cells were purified from the lines by MACS positive selection and used in a sensitive IFN-γ ELISPOT assay to detect single cell cytokine production. The CD8⁺ T cells were stimulated overnight with 2 μM of one of the 33 20-mer overlapping peptides from Ag85A to determine which peptides contained CD8⁺ T cell reactive epitopes.

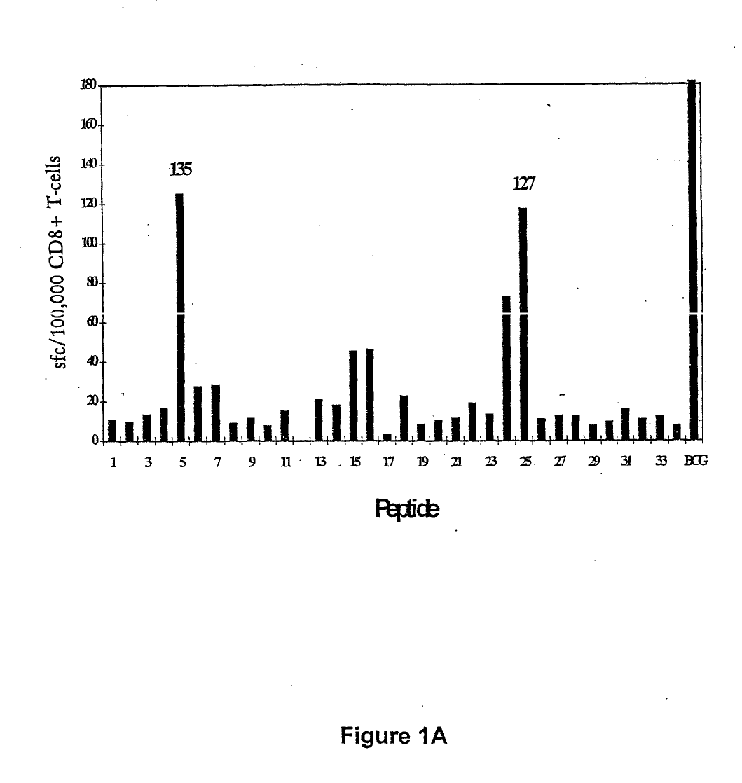

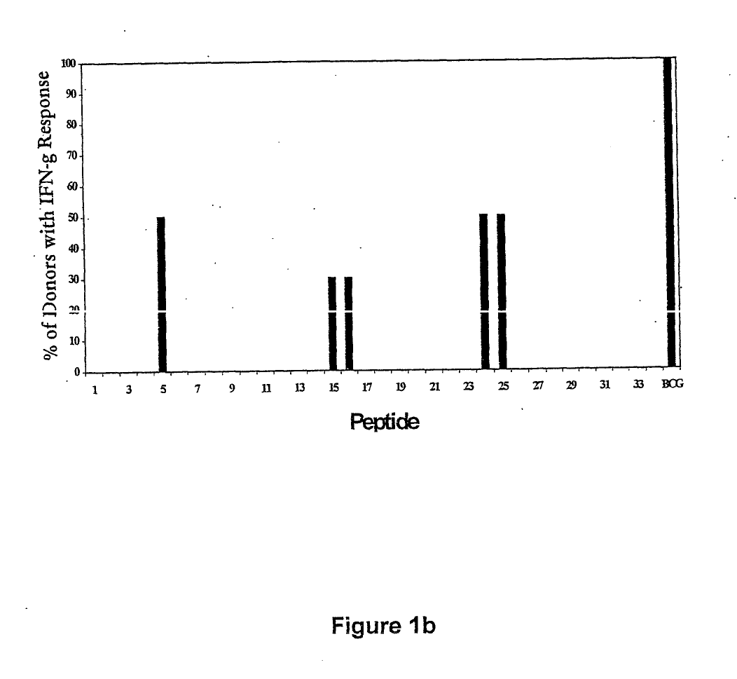

IFN-γ production was observed in response to 5 of the 33 peptides; peptides 5, 15, 16, 24 and 25 (FIG. 1). The strongest responses were observed against p5 and p25 with the average number of spot forming cells (sfc's) per 10⁵ CD8⁺ T cells being 135 for peptide 5, and 127 for peptide 25. Closer sequence analysis of these two peptides revealed HLA-A*201 binding motifs within these peptides; Tissue typing of donors who showed reactivity to these peptides revealed these subjects to be HLA-A*201.

### EXAMPLE 3

IFN-γ Release in Response to M. tuberculosis Derived Peptides

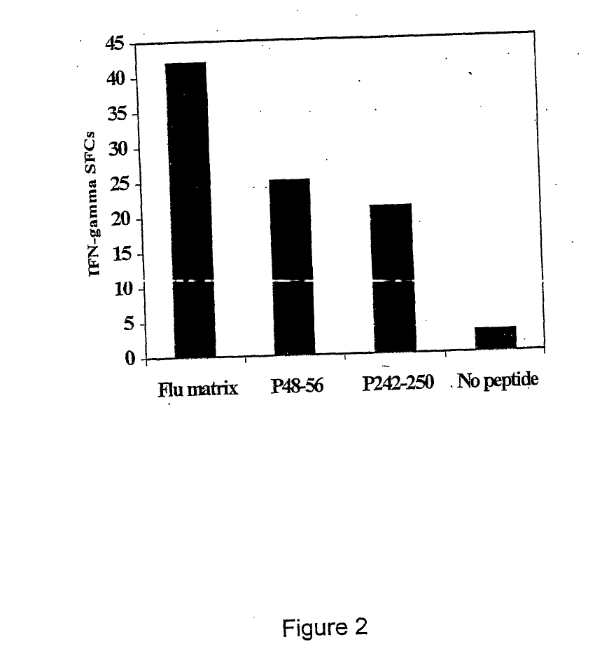

Two CD8⁺ T cell epitopes were identified within p5 and p24/25. To determine the frequency of circulating CD8⁺ T cells reactive to each peptide fresh PBMCs were screened by ELISPOT. Uncultured PBMCs from HLA-A2 donors from whom M. bovis BCG STCLs responded to peptides 5 and 25, secreted IFN-γ in response to the two 9-mers (P₄₈₋₅₆ and P₂₄₂₋₂₅₀) within Ag85A. The mean number of IFN-γ sfc's enumerated from 5×10⁵ PBMCs was 27 for P₄₈₋₅₆ and 22 for P₂₄₂₋₂₅₀ compared with 3 in control wells. The frequency of P₄₈₋₅₆ and P₂₄₂₋₂₅₀ specific IFN-γ sfc's is of the same order of magnitude as SFCs for HLA-A*201-restricted influenza matrix epitope FMP Ml₅₈₋₆₆ (FIG. 2).

### EXAMPLE 4

CTL Response to M. tuberculosis Derived Peptides

To confirm the tissue restriction of these two epitopes 9-mer peptides (P₄₈₋₅₆ and P₂₄₂₋₂₅₀) were used to grow STCL from five HLA-A2 donors. Positively selected CD8⁺ T cells were analysed for their cytotoxic activity against the human HLA-A2 restricted monocyte cell line THP-1 pulsed with peptide. After 2 weeks of stimulation, CTL activity was detected in both cell lines against their relevant peptides.

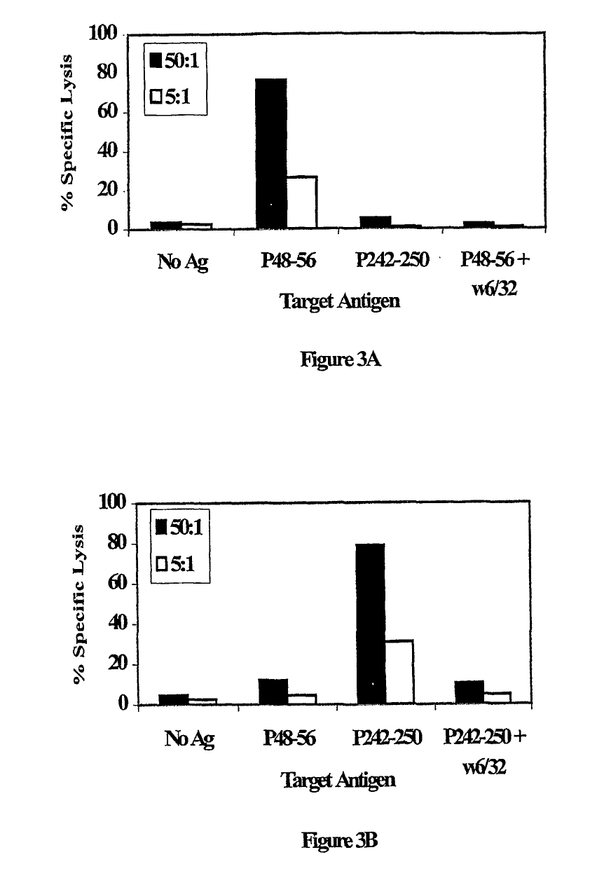

As seen in FIG. 3A, P₄₈₋₅₆ stimulated CD8⁺ T cells showed strong CTL activity against THP-1 cells pulsed with P₄₈₋₅₆ (76% Specific Lysis at 50:1 E:T ratio), but no significant lysis against either FMP M1₅₈₋₆₆ or P₂₄₂₋₂₅₀ pulsed targets (<10% specific lysis at 50:1 E:T ratio). Conversely, P₂₄₂₋₂₅₀ reactive CD8⁺ T cells show potent CTL activity against P₂₄₂₋₂₅₀ pulsed THP-1 cells (79% specific lysis at 50:1 E:T ratio), but no significant lysis against FMP M1₅₈₋₆₆ or P₄₈₋₅₆ pulsed target cells. The cytolytic activity of these Ag-specific CD8⁺ CTLs was completely inhibited by the addition of an anti-MHC class I mAb W6/32 (FIGS. 3A and 3B). Blocking with this mAb reduced the levels of lysis to that observed against uninfected targets. These results demonstrate that the peptide specific CD8⁺ T cells are HLA-A2 restricted.

### EXAMPLE 5

Peptide-Specific CD8⁺ CTLs Recognise Endogenously-Synthesised Ag

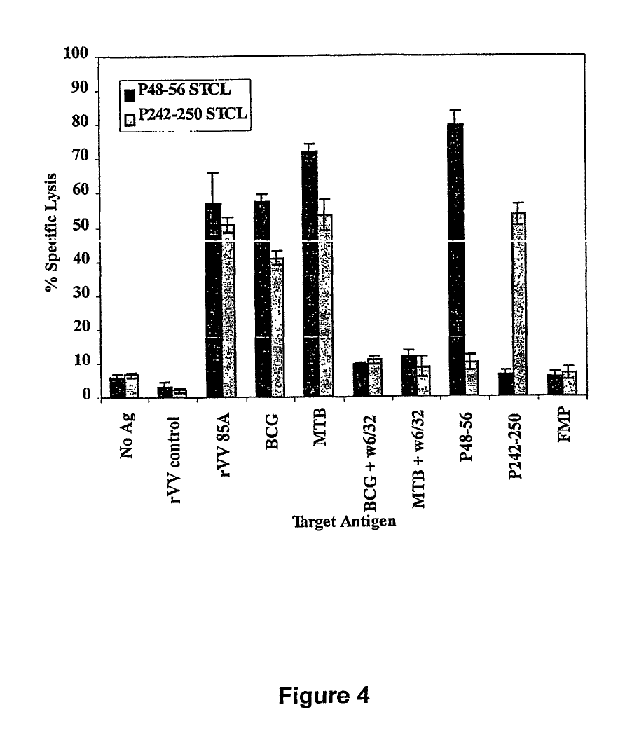

To examine the ability of peptide-specific CTLs to recognise endogenously generated epitopes, autologus monocyte-derved-macrophages were acutely infected with either M. bovis BCG, M. tuberculosis bacilli, or rVV expressing the whole Ag85A protein. FIG. 4 shows that P₄₈₋₅₆ and P₂₄₂₋₂₅₀ specific CTLs are able to recognise and lyse M. bovis BCG, M. tuberculosis, and rVV85A infected macrophages in addition to peptide pulsed targets. Again the T cells were shown to be specific for the peptide used to generate the STCL and there was no recognition of the FMP M1₅₈₋₆₆. The lysis observed against M. bovis BCG and M. tuberculosis bacilli was shown to be MHC class I restricted by blocking with mAb w6/32. These results show that peptide specific CTLs are capable of recognising endogenous generated peptide in a MHC class I restricted manner.

### EXAMPLE 6

Use of Particle Mediated DNA Delivery to Induce HLA-A2-Restricted Peptide Specific CD8⁺ T Cells in a Transgenic Mouse Model

HLA-A2 transgenic C57B1/6 mice were vaccinated according to the following vaccination schedule:

(a) gene gun—the mouse abdomen was shaved prior to gene gun administration of 2 cartridges each containing ˜0.5 μg of DNA encoding M. tuberculosis Ag85A at non overlapping sites i.e. a total of 1 μg; or

(b) intramuscular—50 μl containing 100 μg of DNA encoding M. tuberculosis Ag85A in PBS was administered to the quadriceps muscle; or

(c) intra-dermal BCG—50 μl containing 10⁴ cfu was administered into base of tail; or

(d) subcutaneous peptides—0.2 ml of an incomplete Freund's adjuvant (IFA)/peptide emulsion was divided between 2 sites (right and left flanks). The peptides were formulated at a concentration of 100 μg/100 μl and emulsified 1:1 in IFA using a vortex mixer immediately prior to vaccination. The peptides used were:

P₄₈₋₅₆ and P₂₄₂₋₂₅₀

The mice were vaccinated 3 times at 2 week intervals with the exception of the BCG group where only one vaccination was performed at time 0. Three mice per group were sampled for ELISpot analysis (spleens) 7 days after vaccinations 2 and 3; lymph nodes were also collected from the mice for further analysis.

Spleens and lymph node cells were isolated by physical disruption through a cell strainer. They were re-stimulated in vitro in the presence of the above peptides at 1 μM and IL-2 at 10 ng/ml for 7 days. Cells at 4×10⁵/well were added to ELISpot plates coated with anti-IFNγ antibodies, in the presence of 1×10⁵ irradiated autologous feeders pulsed with peptide. In the case of conA the feeders were not pre-pulsed with peptide and conA was added at 1 μg/ml. Negative controls were in the absence of stimulus. Plates were incubated overnight and spots developed according to standard procedures. Image analysis software was used to count the spots.

At the first time point (7 days post first boost), no IFNγ producing cells were observed in the spleens of any vaccinated animal. However, a response to P₂₄₂₋₂₅₀ was seen in the lymph node of animals vaccinated with the peptides in IFA. This response was HLA-A2 restricted as the peptide was not recognised when feeder cells from wild type C57B1/6 mice were used.

At 7 days post second boost, no IFNγ producing cells were observed in the lymph nodes. However, IFNγ producing T cells were observed in the spleens. DNA vaccination, both via intramuscular and particle-mediated delivery, resulted in significant peptide specific responses. BCG vaccination was less efficient than DNA vaccination in eliciting peptide-specific responses. Peptide vaccination resulted in a P₄₈₋₅₆ response at this time point.

Discussion

M. bovis BCG reactive CD8⁺ T cells were shown to recognise and produce the protective type 1 cytokine IFN-γ in response to five peptides within Ag85A as identified by ELISPOT. The strongest response was observed against peptides 5 and 25, where there appeared to be a non-competitive expansion of T cells reactive towards each of these peptides. A similar non-competitive expansion of T cells reactive to different epitopes was found with Listeria monocytogenes infection in nice. The frequency of CD8⁺ T cells reactive against each of these peptides was ˜1:500 after 14 days of M. bovis BCG stimulation. This number is remarkably high due to the number of secreted proteins produced by M. bovis BCG. Indeed, a relatively high frequency of peptide 5 and 25 reactive CD8⁺ T cells was observed within the circulating PBMC population.

Within the reactive peptides HLA-A2 motifs were noted and used to construct 9-mer peptides (P₄₈₋₅₆ and P₂₄₂₋₂₅₀) of the predicted epitopes alone. These 9-mer peptides were used in ex vivo experiments with PBMC from donors who showed a strong response against each peptide, thus showing that there are circulating CD8⁺ T cells capable of recognising mycobacterial secreted antigens in BCG vaccinated donors. Approximately 1: 18,000 circulating CD8⁺ T cells, were found to be specific for each epitope (P₄₈₋₅₆ and P₂₄₂₋₂₅₀), a number comparable to PBMC specific for the HLA-A*201 restricted influenza virus protein FMP M1₅₈₋₆₆ which gives a frequency of approximately 1:12,000, despite the fact these donors had been BCG vaccinated up to 20 years earlier. Of the two epitopes identified, P₂₄₂₋₂₅₀ appears to be specific for Ag85A, whilst P₄₈₋₅₆ is shared between Ag85A, B, and C, and as a result is the stronger vaccine candidate.

The HLA-restriction of the epitopes identified was confirmed using the HLA-A2 monocyte cell line THP-1 as APCs in a conventional ⁵¹Cr release assay. P₄₈₋₅₆ and P₂₄₂₋₂₅₀ specific CD8⁺ T cell lines recognised THP-1 cells pulsed with the relevant peptide, but did not recognise the irrelevant peptide (i.e. P₄₈₋₅₆ specific lines recognised P₄₈₋₅₆ pulsed targets, but not P₂₄₂₋₂₅₀ pulsed targets, and vice versa) or the influenza HLA-A2 specific peptide FMP M1₅₈₋₆₆. This demonstrated that CD8⁺ T cells were antigen specific and HLA-A*201 restricted. This also demonstrated that in addition to IFNγ secretion, the peptide specific CD8⁺ T cells were potent CTL effector cells.

The question of whether these peptide specific CD8⁺ T cell lines could recognise endogenously generated epitopes was answered by infecting autologous monocyte-derived macrophages with either live bacterial M. bovis BCG/M. Tuberculosis), or recombinant vaccinia virus (rVV) expressing the whole Ag85A protein. The observation that P₄₈₋₅₆ and P₂₄₂₋₂₅₀ specific CTL lines recognise autologous macrophages infected with either rVV expressing Ag85A, M. bovis BCG, or M. tuberculosis demonstrates that this antigen is endogenously processed through the MHC class I presentation pathway, thus resulting in presentation of the epitopes P₄₈₋₅₆ and P₂₄₂₋₂₅₀ through HLA-A2.

In summary, we have identified two CD8⁺ T cell HlA-A*201 restricted epitopes within Ag85A, the major secreted protein of M. tuberculosis. Circulating PBMCs from healthy BCG vaccinated donors recognise these epitopes and produce the protective cytokine IFN-γ at a similar frequency to that of an influenza matrix protein. Peptide specific cell lines possess potent CTL activity against peptide pulsed target cells.

Furthermore, these peptide specific CD8⁺ T cells are capable of recognising endogenously processed and presented antigen from M. bovis BCG and M. tuberculosis infected macrophages. These results indicate that these Ag85A epitopes are particularly suitable peptides for use in a vaccine.

1. Use of (a) a polypeptide which comprises an epitope sequence of formula (I):
X₁ L/I/M X₂ X₃ X₄ X₅ X₆ X₇ V/L/I (I)
wherein
X₁ is G, X₂ is P, X₃ is V, X₄ is E, X₅ is Y, X₆ is L and X₇ is Q, or
X₁ is K or R, X₂ is I or V, X₃ is A, X₄ is N, X₅ is N, X₆ is T and X₇ is R, or an epitope sequence which is an analogue of (I) and which can be recognised by a CD8 T cell that recognises (I); or
(b) an expression vector comprising a polynucleotide encoding a said polypeptide (a) operably linked to a regulatory sequence capable of providing for expression of the said polypeptide (a);
for use in the manufacture of a medicament for vaccinating prophylactically or therapeutically against infection by a mycobacterium by stimulating a CD8 T cell response.

2. Use according to claim 1 wherein the polypeptide has a length of up to 50 amino acids and/or the polypeptide comprises a sequence which is a derivative of the sequence of Ag85A.

3. Use according to claim 1 or 2 wherein the polypeptide comprises two or more copies of said epitope sequence.

4. Use according to any one of the preceding claims wherein the polypeptide also comprises a sequence that enhances the immunogenicity of said epitope sequence.

5. Use according to claim 4 wherein the sequence that enhances immunogenicity is hepatitis B core antigen or a stress protein.

6. Use according to any one of the preceding claims wherein the polypeptide or expression vector is associated with an adjuvant or delivery system capable of stimulating a CD8 T cell response.

7. Use according to any one of the preceding claims wherein the polypeptide or expression vector are in a cell.

8. Use according to claim 7 wherein the cell is a professional antigen presenting cell.

9. A vaccine composition which comprises a polypeptide or expression vector as defined in any one of claims 1 to 8 and an adjuvant or delivery system capable of stimulating a CD8 T cell response.

10. A method of vaccinating a pre-selected host to stimulate a CD8 T cell response against a mycobacterial infection, comprising administering to the host an effective amount of a polypeptide or expression vector as defined in any one of the claims 1 to 8 or a vaccine composition according to claim 9.

11. A polypeptide or expression vector as defined in any one of claims 2 to 8 or a cell as defined in claim 7 or 8.

12. A polypeptide, expression vector or cell according to claim 11 for use in vaccinating against infection by a mycobacterium.

13. A product which selectively binds a T cell receptor that recognises an epitope sequence as defined in claim 1, which product comprises an HLA molecule, or a fragment thereof, comprising a peptide with the sequence of a said epitope sequence in its peptide binding groove.

14. Method of detecting in a population of T cells the presence or absence of CD8 T cells that recognise an epitope sequence as defined in claim 1, said method comprising:
(i) contacting a population of cells comprising CD8 T cells with a polypeptide as defined in any one of claims 1 to 8 or a product as defined in claim 13, and
(ii) determining whether the CD8 T cells recognise said polypeptide or product to determine thereby the presence or absence of CD8 T cells that recognise the epitope.

15. A method according to claim 14 wherein step (ii) comprises detecting the expression of a substance by the T cells, the detection of expression of the substance indicating that the T cells have recognised the said polypeptide.

16. A method according to claim 14 wherein step (ii) comprises detecting lysis by T cells of cells that present the said polypeptide on their surface, the detection of said lysis indicating that the T cells have recognised the polypeptide.

17. A method of diagnosis of a mycobacterial infection or of testing the effectiveness of a vaccination against a mycobacterial infection, said method comprising determining the presence or absence in a host of a CD8 T cell response to an epitope sequence as defined in claim 1, the presence of the CD8 T cell response indicating that the host has a mycobacterial infection or that the vaccination has been effective.

18. A method according to claim 17 wherein the presence or absence of the CD8 T cell response is determined by identifying the presence or absence in a sample from the host of T cells that recognise the epitope sequence using the method of any one of claims 14 to 16.

19. A T cell capable of recognising an epitope sequence as defined in claim 1.

20. A T cell receptor, or fragment thereof, which is capable of binding an epitope sequence as defined in claim 1.

21. A T cell according to claim 19 for use in a method of treating a mycobacterial infection.

22. A method of treating a mycobacterial infection in a pre-selected host, comprising administering to the host an effective amount of T cells as defined in claim 19.


SEQUENCE LISTING (4 sequences)

SEQ ID NO: 1 - protein, 9 aa, Mycobacterium tuberculosis
GLPVEYLQV

SEQ ID NO: 2 - protein, 9 aa, Mycobacterium tuberculosis
KLIANNTRV

SEQ ID NO: 3 - DNA, 1014 bp, Mycobacterium tuberculosis
  CDS (1)..(1014)
atgcagcttgttgacagggttcgtggcgccgtcacgggtatgtcgcgtcgactcgtggtcggggccgtcggcgcggccctagtgtcgggtctggtcggcgccgtcggtggcacggcgaccgcgggggcattttcccggccgggcttgccggtggagtacctgcaggtgccgtcgccgtcgatgggccgtgacatcaaggtccaattccaaagtggtggtgccaactcgcccgccctgtacctgctcgacggcctgcgcgcgcaggacgacttcagcggctgggacatcaacaccccggcgttcgagtggtacgaccagtcgggcctgtcggtggtcatgccggtgggtggccagtcaagcttctactccgactggtaccagcccgcctgcggcaaggccggttgccagacttacaagtgggagaccttcctgaccagcgagctgccggggtggctgcaggccaacaggcacgtcaagcccaccggaagcgccgtcgtcggtctttcgatggctgcttcttcggcgctgacgctggcgatctatcacccccagcagttcgtctacgcgggagcgatgtcgggcctgttggacccctcccaggcgatgggtcccaccctgatcggcctggcgatgggtgacgctggcggctacaaggcctccgacatgtggggcccgaaggaggacccggcgtggcagcgcaacgacccgctgttgaacgtcgggaagctgatcgccaacaacacccgcgtctgggtgtactgcggcaacggcaagccgtcggatctgggtggcaacaacctgccggccaagttcctcgagggcttcgtgcggaccagcaacatcaagttccaagacgcctacaacgccggtggcggccacaacggcgtgttcgacttcccggacagcggtacgcacagctgggagtactggggcgcgcagctcaacgctatgaagcccgacctgcaacgggcactgggtgccacgcccaacaccgggcccgcgccccagggcgcc

SEQ ID NO: 4 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA